(19) United States
(12) Patent Application Publication
(10) Pub. No.: US 2019/0358494 A1
(43) Pub. Date: Nov. 28, 2019

(54) INTERACTIVE EXERCISE EQUIPMENT

(71) Applicant: MCCANN WORLD GROUP PTY LTD, Sydney, New South Wales (AU)

(72) Inventors: Patrick BARON, Melbourne, Victoria (AU); Alec HUSSAIN, Melbourne, Victoria (AU)

(21) Appl. No.: 16/477,737

(22) PCT Filed: Jan. 3, 2018
(86) PCT No.: PCT/AU2018/050003

§ 371 Date: Jul. 12, 2019

(30) Foreign Application Priority Data
Jan. 13, 2017 (AU) 2017900100

(51) Int. Cl. A63B 26/00; A61M 21/02

(52) U.S. Cl. CPC A63B 26/00; A61M 2021/0022; A61M 21/02

(57) ABSTRACT

The present invention relates to equipment used in the exercise of human beings. The equipment may be of a commercial type as used in gymnasiums, or of a consumer type for use in a domestic environment. Exercise apparatus configured such that a mechanical motion of a user executing an exercise activity positively affects a child on or about the apparatus are provided. The exercise apparatus may be configured to translate a mechanical motion of a user executing an exercise activity on the apparatus into a mechanical motion capable of positively affecting a child on or about the apparatus. The positive effect on the may be any one or more of moving, entertaining, occupying, distracting, stimulating, calming, settling, and educating. The mechanical motion capable of affecting a child has the effect of moving a child on or about the apparatus. The exercise apparatus may comprise child physical engagement means configured to receive the translated mechanical motion of the user so as to move a child in or about the child physical engagement means.

### RELATED APPLICATIONS

This application claims priority from Australian provisional patent application 2017900100 filed 13 Jan. 2017.

### FIELD OF THE INVENTION

The present invention relates to equipment used in the exercise of human beings. The equipment may be of a commercial type as used in gymnasiums, or of a consumer type for use in a domestic environment.

### BACKGROUND TO THE INVENTION

Exercise machines are well known in the art of fitness training. These machines typically require a user to expend energy by the movement of various body parts in accordance with the operation of the machine. Diligent operation of the machine over a period of time may lead to the loss of excess weight, increased cardiovascular fitness, or increased strength in the user.

While exercise with such machines provides clear health benefits, their use is not without problems. In particular, exercising with these machines requires dedicated time to be set aside by the user. Often, an exercise machine must be used to effect multiple repetitions, and furthermore multiples sets of repetitions according to a predetermined training regime in order to obtain a desired heath benefit. Moreover, multiple types of machines are used to exercise the various parts of the body. As will be appreciated, the time involved in properly exercising can be substantial, and often to the point of detracting from other important family duties of the user.

Time pressures with individuals having family commitments often leads to such persons neglecting exercise, and sometimes over a period of many years. Over time, such persons may gain weight and become obese or even morbidly obese, with the incidence of related disorders such as hypertension, diabetes, atherosclerosis and the like increasing amongst this cohort.

Muscle tone can also decrease significantly with a lack of resistance training leading to weakness and generally frailty.

It is an aspect of the present invention to overcome or alleviate a problem of the prior art by providing exercise equipment allows individuals having family commitments and limited free time to exercise sufficiently, and without neglecting such commitments.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect, but not necessarily the broadest aspect, the present invention provides exercise apparatus configured such that a mechanical motion of a user executing an exercise activity positively affects a child on or about the apparatus.

In one embodiment of the first aspect, the exercise apparatus is configured to translate a mechanical motion of a user executing an exercise activity on the apparatus into a mechanical motion capable of positively affecting a child on or about the apparatus.

In one embodiment of the first aspect, the positive effect on the child is selected from the group consisting of moving, entertaining, occupying, distracting, stimulating, calming, settling, and educating.

In one embodiment of the first aspect, the mechanical motion capable of affecting a child has the effect of moving a child on or about the apparatus.

In one embodiment of the first aspect, the exercise apparatus comprises child physical engagement means.

In one embodiment of the first aspect, the child physical engagement means is configured to receive the translated mechanical motion of the user so as to move a child in or about the child physical engagement means.

In one embodiment of the first aspect, the child physical engagement means allows a child to be in a seated position.

In one embodiment of the first aspect, the child physical engagement means comprises a footrest and/or a restraint and/or a holding portion.

In one embodiment of the first aspect, the child physical engagement means is configured such the child substantially faces the user while the user is executing an exercise activity on the apparatus.

In one embodiment of the first aspect, the child physical engagement means is configured such that when a child is physically engaged a resistance force is provided against the user executing an exercise activity on the apparatus.

In one embodiment of the first aspect, the exercise apparatus comprises movement input means configured to physically engage with a body part of the user, the movement input means being moved by the body part of the user executing an exercise activity.

In one embodiment of the first aspect, the movement input means is selected from the group consisting of a handle, a bar, a pedal, a chain, a rope, a paddle, a roller, a stirrup, a step, and a tread.

In one embodiment of the first aspect, the movement input means is configured to allow the user to move a body part through a range of motion required for an exercise activity.

In one embodiment of the first aspect, the exercise activity is selected from the group consisting of rowing, cycling, running, walking, jumping, abdominal crunch, bar throw, bar press, butterfly, calf press, shoulder shrug, squat, Smith press, elliptical training, gluteus maximus hamstring raise, leg extension, leg press, chest press, bicep curl, lateral raise, shoulder press, triceps extension, pull up, push up, back extension, calf raise, leg curl, stair training, and thigh adductor training.

In one embodiment of the first aspect, the exercise apparatus comprises a mechanical linkage between the movement input means and the child physical engagement means, the mechanical linkage functioning so as to translate a movement of a user into a movement of the child physical engagement means.

In a second aspect the present invention provides an exercise apparatus comprising a user movement input means configured to be grasped by the hand(s) of a user, the user movement input means allowing the hand(s) of the user to move bi-directionally substantially vertically when grasping, an elongate member connected to the user movement input means, child physical engagement means connected to a region of the member distal to the user movement input means, wherein upon movement of the movement means substantially vertically the member translates that movement to the child physical engagement means such that the child physical engagement means is concomitantly moved along a vertical plane.

In one embodiment of the second aspect, the user movement input means comprises a hand engagement portion, the hand engagement portion being disposed a sufficient vertical distance above the member such that the user assuming a standing position upon a substrate is capable of fully extending his/her arm(s) upwardly or downwardly without the member contacting the substrate.

In one embodiment of the second aspect, the hand engagement portion is at least about 30 cm, 40 cm, 50 cm, 60 cm, 70 cm, 80 cm, 90 cm or 100 cm distal to the point of attachment of the hand engagement portion to the member.

In one embodiment of the second aspect, the user movement input means comprises a hand engagement portion, the hand engagement portion being disposed so as to not overlie any part of the elongate member.

In one embodiment of the second aspect, the hand engagement portion is disposed so as to provide a lateral distance between it and the proximal short edge of the elongate member.

In one embodiment of the second aspect, the lateral distance between the hand engagement portion and the proximal short edge of the elongate member is at least about 30 cm, 40 cm, 50 cm, 60 cm, 70 cm, 80 cm, 90 cm or 100 cm.

In one embodiment of the second aspect, the lateral distance between the hand engagement portion and the proximal short edge of the elongate member is sufficient to allow a user to stand therebetween.

In one embodiment of the second aspect, the user movement input means comprises a hand engagement portion, the hand engagement portion being disposed a sufficient vertical distance above the member such that the user assuming a standing position upon a substrate is capable of fully extending his/her arm(s) upwardly or downwardly without the member contacting the substrate, and the hand engagement portion is disposed so as to provide a lateral distance between it and the proximal short edge of the elongate member.

In one embodiment of the second aspect, the user movement input means is substantially U-shaped.

In one embodiment of the second aspect, the exercise apparatus comprises a pivot axis upon which the elongate member pivots.

In one embodiment of the second aspect, the pivot axis is closer to the end of the elongate member to which the user movement input means is attached so as to increase the work needed to be expended by the user to move the opposing end of the elongate member upwardly.

In one embodiment of the second aspect, the exercise apparatus comprises resistance means configured to resist user movement of the user movement input means.

In one embodiment of the second aspect, the exercise apparatus comprises two or more child physical engagement means.

In one embodiment of the second aspect, the child physical engagement means comprises a seat and/or a handle configured such that a child disposed therein faces a user.

In one embodiment of the second aspect, the exercise apparatus comprises two or more child physical engagement means,

In one embodiment of the second aspect, the exercise apparatus comprises one or more guards configured to inhibit or prevent human contact with area about the pivot axis.

In one embodiment of the second aspect, the exercise apparatus is configured so as to allow a user holding the user movement input means to execute a lat push down exercise activity.

In one embodiment of the second aspect, the exercise apparatus has the features of any one of the embodiments of the first aspect.

In a third aspect, the present invention provides exercise apparatus comprising a user movement input means configured to be grasped by the hand(s) of a user, the user movement input means allowing the hand(s) of the user to move bi-directionally substantially horizontally when grasping, child physical engagement means, a mechanical linkage between the user movement input means and the child physical engagement means configured such that a substantially horizontal movement of the user movement input means is translated into motion of the child physical engagement means.

In a third aspect, the present invention provides the movement input means comprises a hand engagement portion connected to a cable, the cable operably connected to a rotating element such that a user pulling the hand engagement portion causes rotation of the rotating element, the rotating element operably connected to the child physical engagement means such that rotation of the rotating element causes motion of the child physical engagement means.

In one embodiment of the third aspect, the motion translated from the user movement input means to the child physical engagement means is substantially vertical and bi-directional.

In one embodiment of the third aspect, the rotating element is configured as a cam so as to provide the substantially vertical and bi-directional motion to the child physical engagement means.

In one embodiment of the third aspect, the exercise apparatus is configured such that vertical motion is imparted to two points on the child physical engagement means such that one point is travelling upwardly while the other in travelling downwardly so as to provide a substantially undulating motion to the child physical engagement means.

In one embodiment of the third aspect, the child physical engagement means comprises a seat and/or a handle configured such that a child disposed therein faces a user.

In one embodiment of the third aspect, the child physical engagement means is configured to represent an animal, and further configured such that a child has the perception of riding the animal.

In one embodiment of the third aspect, the exercise apparatus comprises one or more guards configured to prevent human contact with any one or more moving part of the apparatus.

In one embodiment of the third aspect, the exercise apparatus comprises a seat upon which a user sits while engaged with the user movement input means, the seat being capable of sliding substantially horizontally.

In one embodiment of the third aspect, the exercise apparatus comprises resistance means configured to resist user movement of the user movement input means

In one embodiment of the third aspect, the exercise apparatus is configured so as to allow a user holding the user movement input means to execute a rowing exercise activity.

In one embodiment of the third aspect, the exercise apparatus has the features of any one of the embodiments of the first aspect.

In a fourth aspect, the present invention provides exercise apparatus comprising a user movement input means configured to be moved by the arms(s) of a user, the user movement input means allowing the arms(s) of the user to move bi-directionally about the shoulder joint(s), and child physical engagement means, wherein the child physical engagement means is attached to the apparatus and configured such that a child disposed in the child physical engagement means substantially faces the user while the user is executing an exercise activity on the apparatus.

In one embodiment of the fourth aspect, the exercise apparatus comprises an arm extending from an upper region of the apparatus and outwardly over the head of a user disposed on the apparatus, wherein the child physical engagement means is a seat suspended from the arm in a swing-like manner.

In one embodiment of the fourth aspect, the arm is flexible about its long axis such that movement of the user in the execution of an exercise activity is translated to the arm to cause minor bending of the arm so as to in turn impart a jiggling and/or swinging motion to the child physical engagement means.

In one embodiment of the fourth aspect, the exercise apparatus comprises a cable running along the arm, the first end of the cable operably connected to the user movement input means associated with the arm which operatively connects the user movement input means, and the second end of the cable operably connected to the child physical engagement means, the operable connections being such that movement of user movement input means in the execution of an exercise activity imparts movement onto the child physical engagement means.

In one embodiment of the fourth aspect, the user movement input means comprise one or more paddles configured to contact the user's arm(s) and against which the user arm(s) exert a force in the execution of an exercise activity.

In one embodiment of the fourth aspect, the user movement input means is/are configured such that the paddles alternately conceal and reveal a user's face to a child disposed in the child physical engagement means in the execution of an exercise activity.

In one embodiment of the fourth aspect, the user movement input means is/are configured such that in the execution of an exercise activity the user's arm(s) move from a lateral position substantially medial position with reference to the user's body.

In one embodiment of the fourth aspect, the exercise apparatus of comprises resistance means configured to resist user movement of the user movement input means.

56. The exercise apparatus of any one of claims configured so as to allow a user holding the user movement input means to execute a chest fly exercise activity.

In one embodiment of the fourth aspect, the exercise apparatus has the features of any one of the embodiments of the first aspect.

In a fifth embodiment, the present invention provides an exercise apparatus substantially as hereinbefore described by reference to any of the drawings.

In a sixth aspect, the present invention provides an exercise facility comprising any two or more different exercise apparatuses, each of the two or more exercise apparatuses according to any one of the apparatuses of the first aspect, or the second aspect, or the third aspect, or the fourth aspect, or the fifth aspect.

In one embodiment of the sixth aspect, the exercise facility comprises instructions to the method steps of: a user placing a child into the physical child engagement means of the apparatus, and the user exercising using the apparatus.

### BRIEF DESCRIPTION OF THE DRAWINGS

FIGS. 1 and 2 are diagrammatic representations all of which show preferred embodiment #1 as described in the detailed description of preferred embodiments section herein.

FIGS. 3 and 4 are diagrammatic representations all of which show preferred embodiment #2 as described in the detailed description of preferred embodiments section herein.

FIGS. 5 to 7 are diagrammatic representations all of which show preferred embodiment #3 as described in the detailed description of preferred embodiments section herein.

### DETAILED DESCRIPTION OF THE INVENTION

Reference throughout this specification to “one embodiment” or “an embodiment” or similar wording means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and from different embodiments, as would be understood by those in the art.

In the claims below and the description herein, any one of the terms “comprising”, “comprised of” or “which comprises” is an open term that means including at least the elements/features that follow, but not excluding others. Thus, the term comprising, when used in the claims, should not be interpreted as being limitative to the means or elements or steps listed thereafter. For example, the scope of the expression a method comprising step A and step B should not be limited to methods consisting only of methods A and B. Any one of the terms “including” or “which includes” or “that includes” as used herein is also an open term that also means including at least the elements/features that follow the term, but not excluding others. Thus, “including” is synonymous with and means “comprising”.

Moreover, it is not presented or suggested that any embodiment of the invention described herein has all advantages described herein, or indeed any advantage whatsoever. Some embodiments of the invention may represent only useful alternatives to the prior art.

The present invention is predicated at least in part on Applicant's discovery that a child (including an infant) can become an integral part of a carer's exercise routine where an appropriately configured exercise apparatus is provided. In this way, a parent may remain engaged with his/her child while exercising, the parent gaining the benefits of exercise while the child gains the benefits of parental interaction. There is no need for the parent to wait until the child is asleep to commence exercising, or to bear the constant interruptions that a child may cause to an exercise routine as a result of being ignored. In particular, Applicant proposes an exercise machine that is capable of positively affecting the child. Accordingly, in a first aspect the present invention provides Exercise apparatus configured such that a mechanical motion of a user executing an exercise activity positively affects a child on or about the apparatus.

The child is typically disposed on or about the apparatus, and is generally supported and/or retained in a position (often being a seat or the like in physical connection with the apparatus) so as to achieve the positive effect. In one embodiment, the apparatus is configured to exploit the work performed by the parent in the course of exercising on the apparatus in order to achieve the positive effect. In this way, the child is automatically positively affected by the parent's exercise routine. This is in direct contrast to prior art exercise machines which (for safety reasons at least) require children to be kept well clear due to the danger of being hit or crushed by moving parts such as moveable handles, weights, cables and the like. The child and parent become emotionally disconnected during the exercise regime which may extend for an hour or more. In this time, the child is likely to become bored or even anxious leading to negative behaviours such as crying and whining. In such circumstances, the parent will typically cease exercise so as to settle the child.

The present invention provides a synergistic effect in that the action of exercising has positive health benefits for the parent (because he/she is left to exercise uninterrupted, is less stressed during exercise, is able to positively engage with the child during exercise, feels less guilt during exercise etc), and also the child (because he/she has a healthier, happier and more energetic parent with all the attendant developmental benefits thereof, and is not left to sit unstimulated or unengaged for the extended period of parental exercise.

In one embodiment, the apparatus is configured to translate a mechanical motion of the parent executing exercising into a mechanical motion that can positively affecting the child. The translated mechanical motion may or may not physically move the child. In the former circumstance, the child may be rocked, jiggled, swayed, rotated, moved upwardly and downwardly, moved left to right, and the like in order to confer a positive outcome on the child. In the latter circumstance, the translated mechanical motion may leave the child stationary but move some other object such as jiggle a soft toy, turn the pages of a brightly coloured flip book, or move a mobile for example. Some forms of the invention are capable of moving both the child and another object.

In any event, the positive effect on the child as provided by the operation of the present apparatus may be any effect that engenders a physical or emotional state in the child which allows the parent to continue to exercise uninterrupted and/or decreases a negative behaviour in the child. The apparatus may be configured to move, entertain, occupy, distract, stimulate, calm, settle or educate the child.

Typically the apparatus has some means for physically engaging the child such that child remains supported or retained in a substantially fixed position. This has the dual effect of prevent the child from moving about and sustaining an injury from the moving parts of the apparatus, and furthermore, maintaining the child in a position or orientation to potentiate the positive effect. For example, the child is preferably oriented so as to face the parent during exercise, this providing an emotional connection for both parent and child. The parties may converse, sing together, exchange comical facial expressions or noises, with such activities being generally entertaining for both parent and child.

In some embodiments, the means for physically engaging the child retains or supports the child in a comfortable manner so as to lessen the probability of negative behaviours in the child. Often a soft or moulded seat is provided for this purpose, the seat typically being mounted directly or indirectly on the apparatus. A backrest may be provided so as to maintain the child in an upright position and to orientate the child in a desired direction, for example toward the parent or other item of interest on or about the apparatus such as a flip book. Where a child is liable to slump or attempt escape from the seat then a belt or strap of some type may be provided. Other means to physically engage the child are anticipated to be useful including a “baby walker” type of apparatus (modified to stay in a fixed position), a suspended “bouncer” type of apparatus, a “porta-cot” type of apparatus, and the like. Foot rests and handles may be provided in some forms of the physical engagement means, and particular where a motion imparted on the child is particularly vigorous. The skilled person having the benefit of the present specification will be enabled by routine means only to conceive of other suitable physical engagement means useful in the context of the present invention.

Some embodiments of the invention comprise mechanical linkage such that an exercise movement of the parent in respect of the apparatus is translated so as to impart movement on the child directly, or more commonly indirectly via the child physical engagement means. Such mechanical linkages are typically positioned operably between a movement input means of apparatus and the child physical engagement means. The skilled person is familiar with movement input means of prior art exercise machines, knowing such means includes handles, bars, pedals, treads and the like. For the avoidance of doubt a movement input means is a component adapted to engage with a body part of a user, with the user pushing and/or pulling the component against some resistive force provided by the apparatus. For example, a bicycle-like exercise machine provides pedals against which the user's feet push in order to turn a crank. As another example, a rowing machine provides a horizontal bar which is grasped by both hands which exert a pulling force so as to tug on a cable attached to a weight by pulley means.

In any event, the mechanical linkage for translating the exercise movement of the user into a movement of the child may comprise any one of more of a cable, a drive shaft, a push rod, a pulley, a joint (such as a universal joint) a hinge, a pivot, a cam, a fulcrum, a lever, a gear, a spline, a flywheel, and the like.

The entire apparatus is generally configured such that the movement input means allows the user to execute the full range of movement required by the exercise for which the apparatus is adapted. For example, where the exercise is a lat push down the apparatus allows the bar which is held by the user to be locatable around the user's upper chest region and then moved downwardly in an unimpeded manner such that the arms finish the motion in substantially extended (straight) form. As another example, where the exercise is a biceps curl the bar which is held by the user is able to be firstly locatable around the user's waist region and then moved upwardly in an unimpeded manner such that the arms finish the motion in substantially flexed (bent) form.

As will be appreciated, the benefits of exercise are amplified where some resistance against movement is provided by an exercise machine. Exercise machines of the prior art typically provide such resistance by having weights which must be moved upwardly against the force of gravity by the user. In some embodiments of the present apparatus, an additional benefit is provided in that the weight of the child acts as a means of resisting user movement. Children gain weight over time, and so the parent's exercise intensity can be naturally, gradually and inevitably increased over time in accordance with their child's increasing weight. Many individuals do not routinely increase the resistance on an exercise with the present invention removing the discretion to do so thereby compelling the parent to increase exercise intensity. It is important to recognise this advantage is synergistic with other advantages of parental/child engagement, positive effects on the child, and uninterrupted exercise session for the parent at least for some embodiments of the apparatus.

In some embodiments, additional resistance means to that provided by the child are provided. Such means include weights, pneumatic means and frictional means. The additional resistance means will typically be adapted to provide various levels of resistance by means well known to the skilled artisan.

The present invention will be more fully described by reference to the following non-limiting embodiments of the present invention.

### PREFERRED EMBODIMENTS OF THE INVENTION

Three preferred embodiments are described below to illustrate more particularly describe the general principles of the invention as described supra, and defined by the claims infra. Each of the preferred embodiments in this section may also be considered as discrete inventions on their own in light of the prior art and therefore potentially severable from each other.

### Preferred Embodiment #1: Lat Push Down Machine

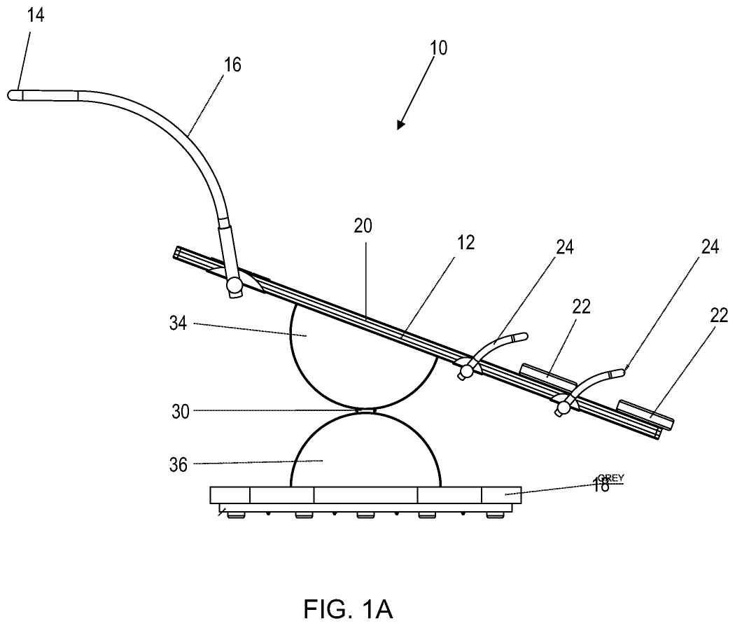

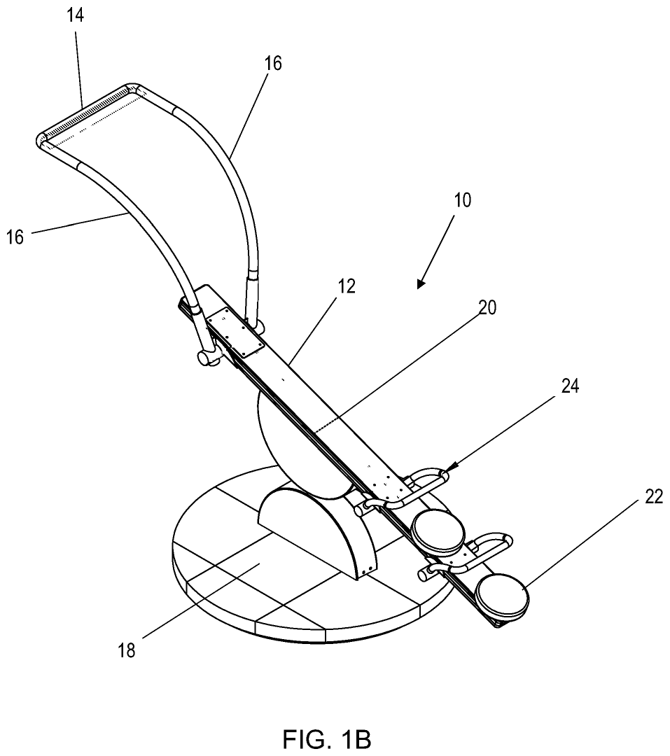

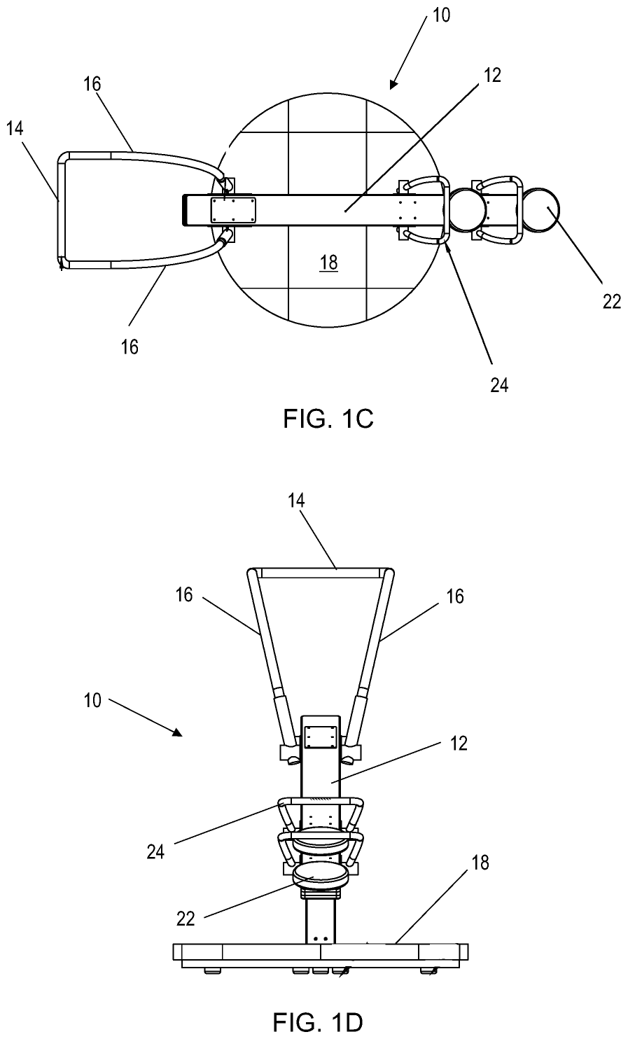

Reference is made to FIG. 1A which shows an exercise machine 10 useful for executing a lat push down exercise. An elongate member 12 having a bar 14 (being a holding portion) attached toward the end of the member 12. As will be more clear from the perspective view shown in FIG. 1B the bar 14 runs horizontally thereby allowing the user to place two hands on the bar. The bar 14 assumes an elevated position by virtue of the arced support arms 16. The arc-shape of the support members 16 further disposes the bar away from the member 12, thereby providing significant clearance between the bar 14 and the closest end of the member 12.

The elevated position and the clearance from the member 12 allows a user holding the bar 14 to execute the full range of motion required to properly perform a lat push down exercise. As will be more fully described infra, the member pivots such that the ends of the member 12 move generally upwardly and downwardly in a reciprocal manner. Thus, the bar 14 being attached to the member 12 allows for a downward movement starting from the elevated position shown in FIG. 1A to a lower position, and finishing at a lower point. The lowest point possible occurs where the end of the member 12 collides with the mounting platform 18 below, although in practice this will not typically be achieved.

In executing a lat push down exercise the user stands to the left of the bar 14 (as drawn) and orientates the shoulders substantially parallel to the bar 14, grasps the bar 14 with the palms facing generally downwardly. It will be noted in this preferred embodiment that the bar 14 is relatively wide, and certainly wider than the member 12, this being most clearly shown in the perspective view of FIG. 1A. It will be noted that the support arms 16 splay outwardly so as to accommodate the width of the bar 14. Provision of a relatively wide bar allows for the user to assume a relatively wide grip (shoulder width, or greater) as is often required in a lat push down exercise.

Once properly grasped, the user then pushes the bar 14 downwardly until the arms are substantially extended straight and generally downwardly from the shoulders. The user then relaxes and allows the bar 14 to rise back to the original position, at which time the user then executes a further downstroke, and so on.

The member 12 pivots about the axis 20 such that as the user pushes downwardly on the bar 14, the right hand end (as drawn) of the member 12 moves generally upwardly. As will be noted this end of the member 12 has two seats 22 disposed thereon. One or both seats 22 (being child physical engagement means) may be occupied by a child, and each child may hold the child handles 24 (also child physical engagement means) for stability. The child(ren) occupying the seat(s) bear their weight downwardly on the right hand end of the member 12 thereby providing a resistance to the user pushing the bar 14 downwardly during the exercise. In this way, the presence of the child(ren) improves the exercise for the parent/user in so far as more energy is expended by the parent, more body fat will be lost, and a greater improvement in muscle tone will result. Concomitantly, the child(ren) is/are moved upwardly and downwardly in the course of the exercise and are entertained and stimulated by the movement. Moreover, the parent/user is facing his/her child(ren) for the duration of the exercise and can interact with them in a positive manner.

It will be immediately appreciated that this embodiment of the invention is very different to a prior art “teeter-totter” (or “see-saw”) which is devoid of the specially configured user movement input means (being the combination bar 14 and support arms 16). A prior art teeter-totter is not designed for a parent to execute a defined exercise, and certainly not a lat push down exercise as provided by this preferred embodiment of the present invention.

At best, a prior art teeter-totter will have a child handle (similar to that marked 24 in the present drawings) which the parent may grasp while pushing down on one end of the teeter-totter so as to elevate the other end upon which a child sits. The holding portion of the child handle in prior art equipment is not elevated and disposed rearwardly so as to provide vertical and horizontal clearance from the end of the main teeter-totter member so as to allow a user to perform the full range of motion required execute a lat push down exercise. A parent attempting a lat push down exercise with prior art playground equipment will firstly experience collision of the end of the teeter-totter with his/her body due to the child handle being positioned inwardly and overlying the main member.

Furthermore, the child handle in prior art equipment will be narrow (being no wider that the seat) preventing an adult user from properly spacing his/her hands on the handle. In the present invention the bar 14 is preferably at least about shoulder width of an average adult male or average adult female. Widths of greater than about 20, 25, 30, 35, 40, 45, 50, 55, 60, 65, 70, 75 or 80 cm are contemplated to provide sufficient width of bar to accommodate an adult wishing to adopt a shoulder width grip or even a broader grip.

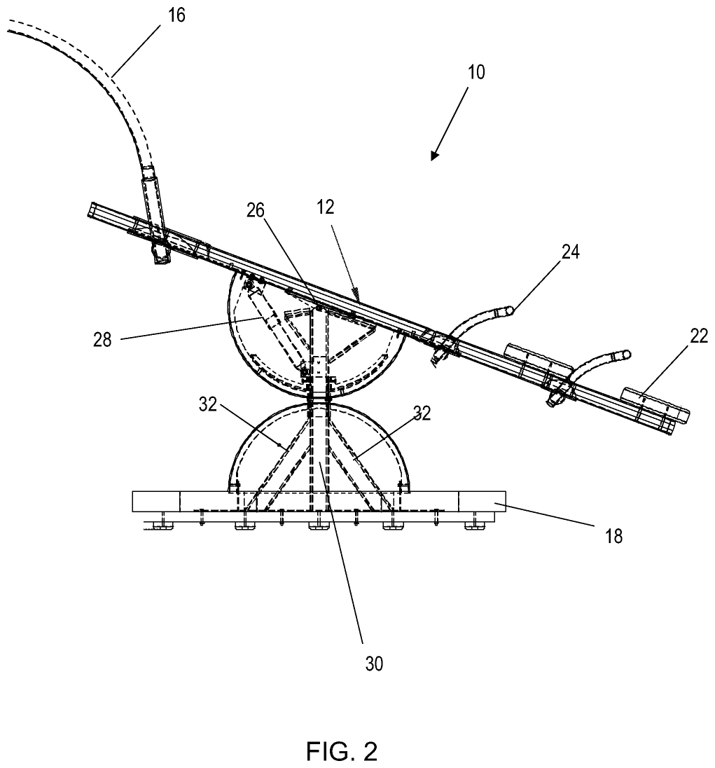

FIG. 2 shows detail of the pivoting mechanism. It will be noted that the member 12 pivots about an underlying pivot pin 26. Relevant to the use of this preferred embodiment as exercise equipment, it will be noted that the pivot axis is disposed not in the centre of the member 12 but more so toward the end having the support arms 16 and bar 14 attached (the left-hand end, as drawn). As will be apparent from basic engineering principles, this arrangement increases the effort required by the parent/user to push the bar 14 downwardly for any given upward movement against gravity of the opposing end upon which a child is disposed, and acting as a counter weight. By this arrangement, the relevant muscles of the parent/user experience significant resistance and considerably more than that provided where the pivot axis is central to the member 12.

Again, this is a very different arrangement from a prior art teeter-totter where the pivot axis is central to the main member such that both ends require the same amount of downward force to be applied so as to move the opposing end upwardly.

This preferred apparatus further comprises a piston-type damper 28 extending laterally and upwardly from the main mounting member 30 of the apparatus to the underside of the member 12. The damper 28 may function so as to increase the resistance faced by the parent/user executing a lat push down exercise. In that function, the damper may be user adjustable to increase or decrease the level of resistance. This modulation of resistance may be used alone or in addition to that provided by movement of the pivot axis as described supra.

In addition or as an alternative to the foregoing, the damper may function to prevent any rapid movement of the member so as to prevent injury to a child seated on the apparatus. In the absence of damper 28 upon a rapid downward movement of the bar 14 by the parent/user the child may be catapulted from the seat 22. Similarly, if the parent/user does not properly control the rate of movement of the bar 14 on the upstroke, the opposing end of the apparatus carrying the child may crash to the ground leading to jarring of the child's back and neck.

The member 12 is generally supported by a single mounting member 30 which is attached to a circular platform typically by bolt means. The member 30 is stabilized by braces 32.

To prevent a child from inserting fingers about the pivoting area of the apparatus, an upper guard 34 is disposed on both sides of the pivot axis 20. A lower guard 36 is also provide to prevent a child climbing under the machine.

### Preferred Embodiment #2: Rowing Machine

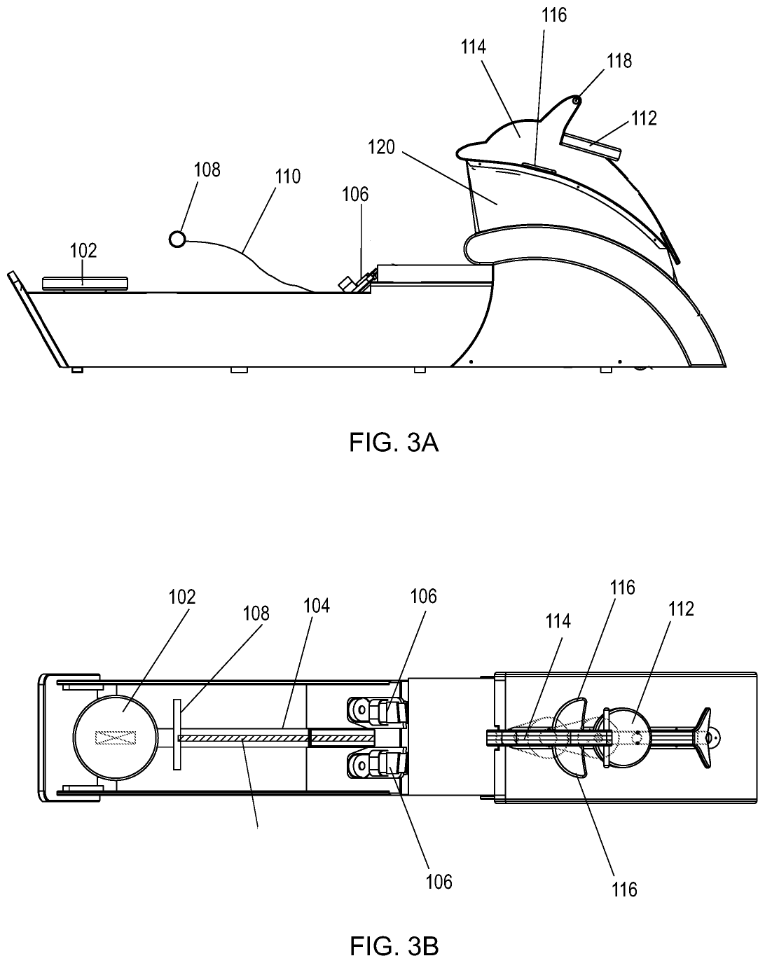

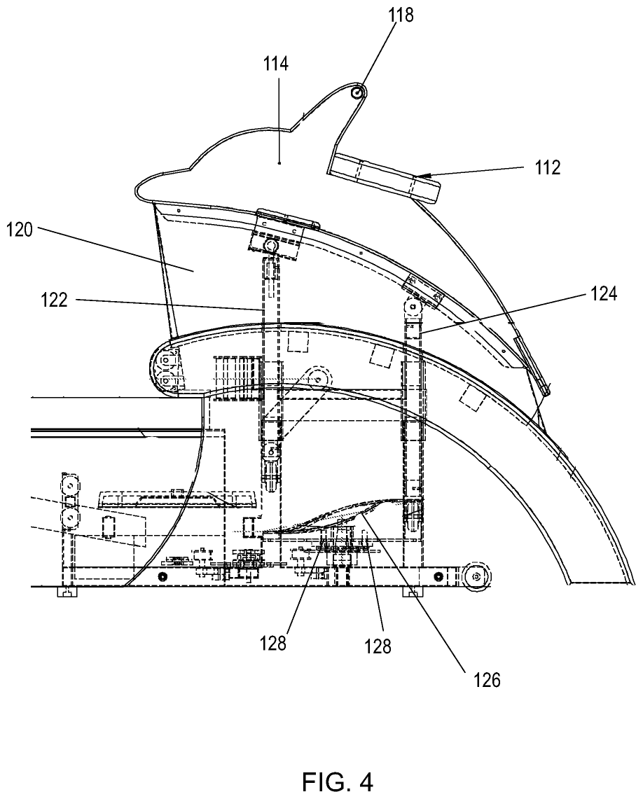

Reference is made to FIG. 3A which shows an exercise machine 100 useful for executing a rowing exercise. The machine provides a seat 102 which slides along a track (104, as shown in the plan view of FIG. 3B). The seat 102 is occupied by the parent/user of the machine.

The parent/user places his/her feet on the footrests 106 and grasps the bar 108 as attached to a first end of the webbing 110. The second end of the webbing 110 passes into to a mechanism for translating the movement of the bar as occurs in the course of a rowing exercise, as will be described more fully infra. Once the webbing 110 is pulled taught by the parent/user, the webbing 110 is further pulled so as to actuate the translation mechanism. Actuation of the translation mechanism in turn has the effect of imparting a rocking motion to the seat 112 (being a physical child engagement means). It will be noted that the seat 112 is disposed upon the representation of a dolphin 114 with two footrests 116 extending from the dolphin 114, the footrests 116 having the appearance of the dolphins pectoral fins. A handle bar 118 is inserted through the dorsal fine of the dolphin 114 to allow the child to grasp. In this context any one of more of the seat 112, footrests 114 and handle bar 118 may be considered the physical child engagement means according to the invention. It will be noted that the position of the handlebar 118 and footrests 116 orientate the child toward the parent/user sitting on the seat 102 and performing a rowing exercise activity.

As for preferred embodiment #1, this embodiment has the advantage of parent/child interaction so as to keep the child entertained during the parent's exercise regime. The child is further entertained by the experience of riding on an artificial dolphin which moves in an undulating motion similar to an animal diving into and out of the water.

Given that moving parts (as will be more fully discussed infra) a disposed beneath the seat, a canvas guard 120 is disposed about the base of the dolphin 114 to prevent any crushing injury to a child's finger inserted into the mechanism.

The relative dispositions of the various components already described may be further understood by reference to plan view of the machine 100 as shown in FIG. 3B.

Turning now to a description of the mechanism for translating the parent/user rowing motion into the dolphin rocking motion, reference is made to FIG. 3C. It will be noted that the dolphin 114 is mounted on two posts 122 (disposed forward from the seat 112) and 124 (disposed rearward to the seat 112) both of which are hingedly connected to the dolphin. The two posts 122 and 124 are movable vertically, and in a reciprocal manner such that (at least for part of a single cycle of operation) when the post 122 is moving upwards, the post 124 is moving downwards. This motion provides an exhilarating ride sensation for the child.

The reciprocating motion is afforded by a rotating plate 126 which functions as a cam mechanism acting to alternately lift and lower each support post 122 and 124 as it turns.

The rotating plate 126 is rotated by way of frictional engagement with the web 110. The rotating plate 126 has disposed thereunder a circumferential face against which the web 110 bears, with close frictional contact between the web 110 and circumferential face being provided by a series of rollers (two of which are marked 128) which urge the web 110 against the circumferential face. Thus, as the web is pulled through the translation mechanism by the user executing the pull-back portion of a rowing action, the rotating plate 126 is caused to rotate.

As will be appreciated, work is required to rotate the plate given the weight bearing thereon by the child (the weight being transferred indirectly via the seat 112 and dolphin 114). Thus, the presence of the child (and in some cases two children may fit on the seat 112) increases the resistance against which the parent/user must exert so as to complete the full rowing action.

A return mechanism (not shown) functions to recoil the web 110 when the pull-back motion is complete and the user slides forward on the seat 102 toward the dolphin 114. Once recoiled, the web 110 is ready to again be pulled taught and through the translation mechanism for the next pull-back motion initiated by the user.

### Preferred Embodiment #3: Pectoral Fly Machine

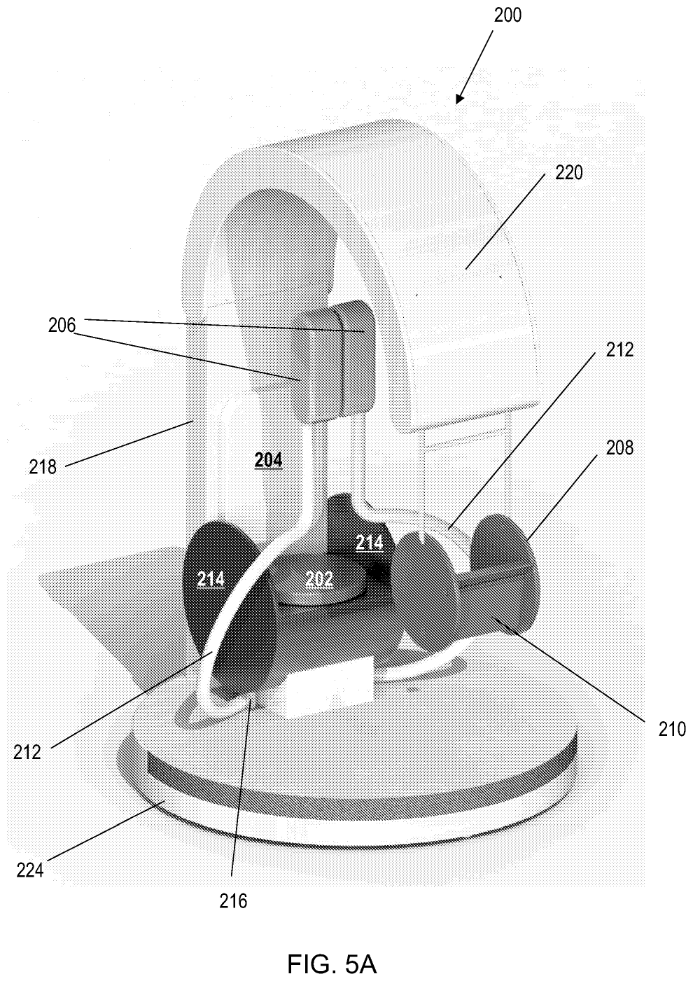

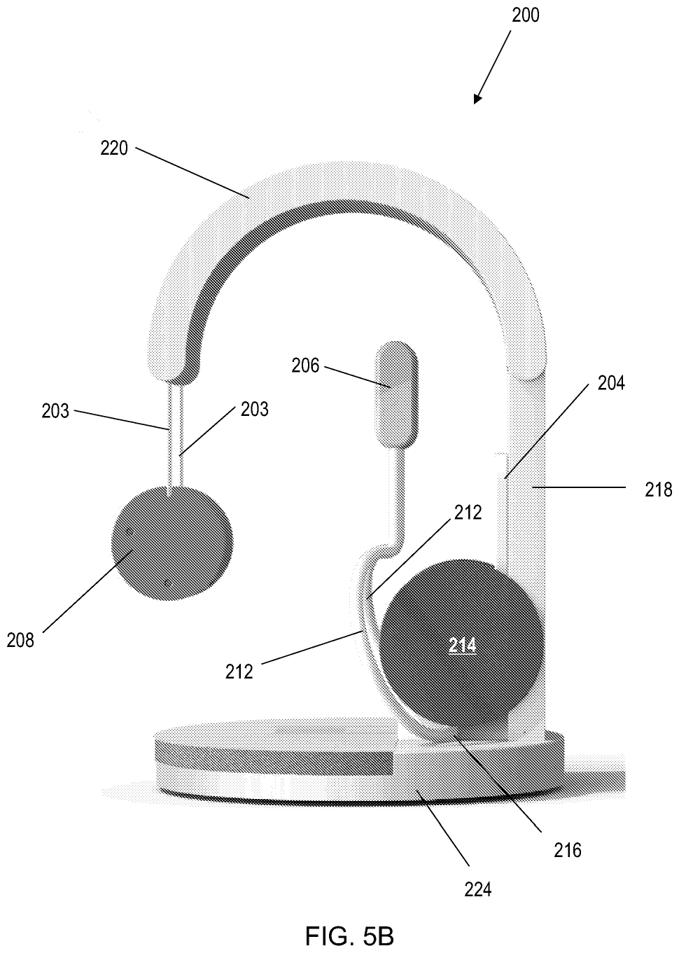

Reference is made to FIG. 5A which shows an exercise machine 200 useful for executing a pectoral fly exercise. To prepare for the exercise, the parent/user sits in the seat 202 with the back against the backrest 204 and places the forearms against the pads 206, the arms generally bent at the elbow. The child is placed in the swing 208 (typically by the parent, and before the parent sits in seat 202), which is suspended by ropes 203.

In executing the exercise, the parent/user rotates the arms inwardly (by rotation of the shoulder joint) so as to cause the pads 206 to come together. This intermediate positions of the pads 206 in the fly exercise are shown in FIG. 5A. In moving from the arms from the lateral position to medial position the pectoralis major muscles are effectively exercised. The arms of the user parent are then rotated in the reverse direction so as to return to the lateral positions. This is done in a controlled manner so as to maintain some tension on the pectoral muscles and further increase the amount of work expended by the parent/user.

As will be noted, the swing 208 has a backrest 210 which acts to support the child, and also to orientate the child so as to face the exercising parent/user. Quite apart from any mechanical motion translated to the swing 208 by the movement of the pads 206 (more of which will be described infra) the movement of the pads 206 in the course of the exercise activity acts to alternately conceal and reveal the parent/user's face to the child. As will be noted, the pads are positioned at sufficient height and dimensioned at sufficient size so as to accomplish concealment of the parent/user's face when the pads 206 are brought together (as shown in FIG. 5A). Thus, a “peek-a-boo” game is established between the parent and child in the course of the exercise activity. This game confers a positive effect by entertaining the child. This advantage is provided because of the arrangement of the swing 208 (being a physical child engagement means) so as to suspend the child's face at substantially the same level as that of the parent/user, and furthermore the orientation of the child toward the parent/user.

To more fully describe this embodiment, a side view of the apparatus of FIG. 5A is shown as FIG. 5B

This preferred embodiment is further configured such that a mechanical movement of the apparatus in the course of an exercise activity is translated into a movement which positively affects a child disposed in the seat 208. It will be noted that each of the pads 206 are connected to an arm 212. Each arm bows outwardly so as to clear the seat arms 214 before attaching to a lower rotating portion (one of the two rotating portions being marked 216). A cable (not shown in this drawing) is operably attached to each rotating portion, with each cable being pulled when the user rotates the pads 206 medially and released the pads 206 are rotated laterally. The two cables run through the interior of the rear support 218, and then through into the interior of the arced swing support 220, terminating at a swing actuating mechanism (not shown) disposed within a cavity of the arced support 220.

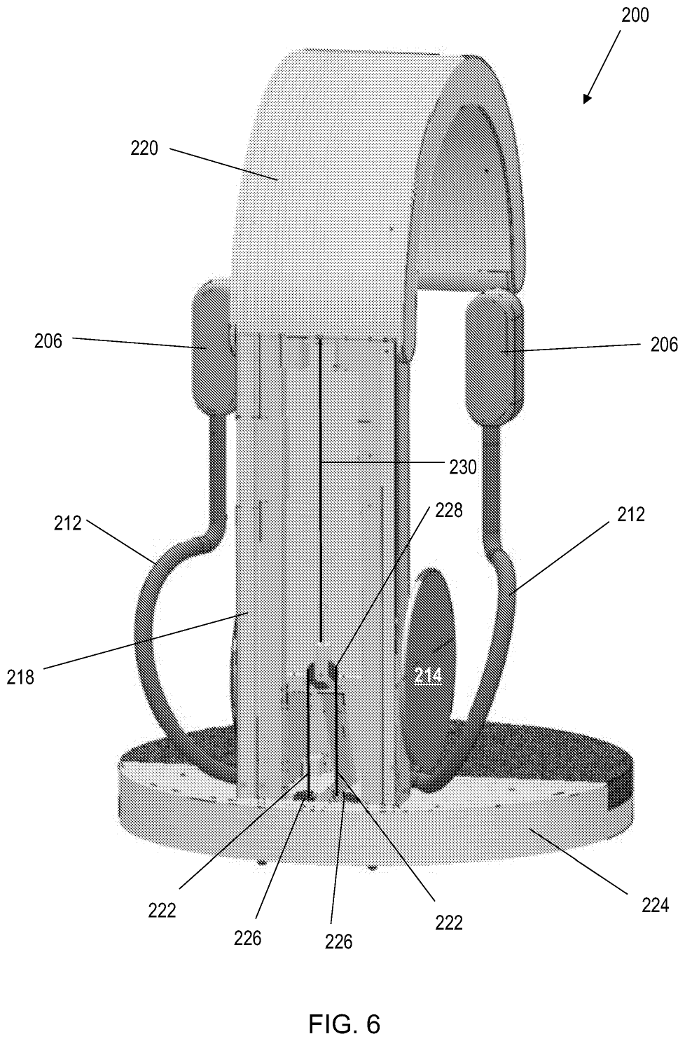

To further explain the mechanism by which the exercise movement is translated to the swing actuating mechanism, reference is firstly made to FIG. 6 which is a rear view of the apparatus 200, and having a rear maintenance cover removed. Removal of the cover exposes two cables 222, each of which is operably attached to its proximal arm rotating portion (not shown in this drawing, but marked 216 in FIGS. 5A and 5B). The operable attachment occurs within a cavity of the base 224 via pulleys 226.

The operable attachment is configured such that rotation of the arms 212 in the course of exercise, causes rotation of the rotating portions 216 at the bottom of the arms 212. The rotating portions act to take-up each of the cables 222 respectively in a spool-like manner, thereby effectively pulling the cables outwardly and through the pulleys 226. The cables 222 loop over the unifying pulley 228 which in in turn moved downwardly. The unifying pulley 228 is connected to cable 230, which is also pulled downwardly. The cable 230 runs upwardly and into the arced swing support 220.

It will be noted that the apparatus shown in FIG. 6 is devoid of the swing 208, 203; this being deliberate so as to improve clarity of the drawing.

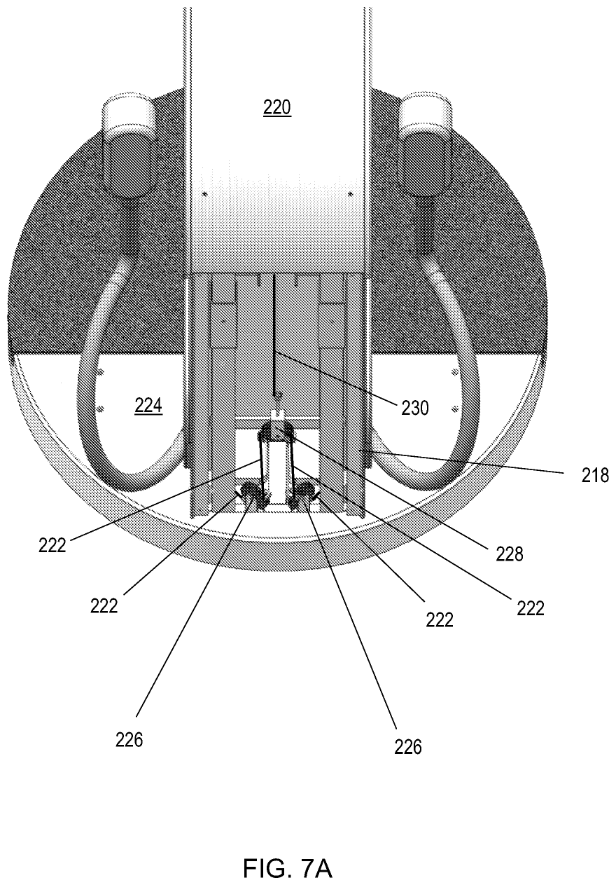

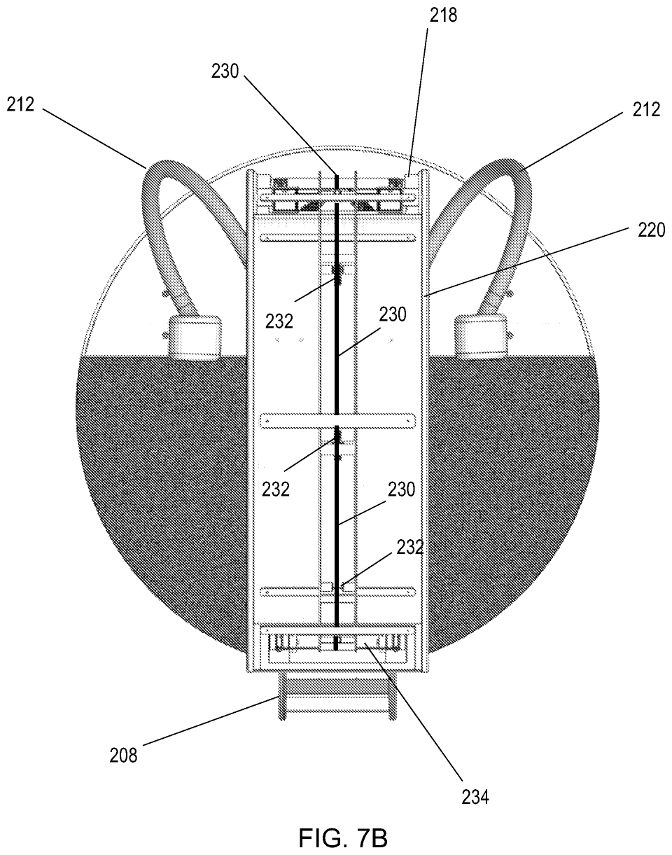

Reference is now made to FIG. 7A which is a plan view of the apparatus as shown in FIG. 6, this view more clearly showing the arrangement of the pulleys 226 about the base 224.

Reference is made now to FIG. 7B which shows a plan view of the apparatus of FIG. 6 albeit with an upper maintenance cover removed. This view reveals the cable 230 extending from the rear support 218 through the arced swing support 220 over a series of pulleys 232 and terminating at the swing actuation mechanism 234. As the cable 230 is pulled over the pulleys (in the upward direction, as drawn), this causes actuation of the mechanism 234 leading to movement of the swing

### Preferred Embodiment #4: Exercise Facility with Combination of Two or More Machines

An exercise facility is provided so as to provide parents and carers the opportunity for exercise without the need to place their child into care, or to ignore the child during an exercise session. The facility has two or more exercise machines according to the present invention, each machine dedicated to a different exercise activity. For example, the facility may have installed two or three exercise machines according to any of preferred embodiments #1, #2 or #3 as described supra.

While the machines will provide the user with a range of exercise activities, in one embodiment of the facility the machines are configured to provide different positive effects to the child as the parent moves from one machine to another. In this way, the child in unlikely to get bored over an entire exercise session which may last for over an hour in some circumstances.

In the description provided herein, numerous specific details are set forth. However, it is understood that embodiments of the invention may be practiced without these specific details. In other instances, well-known methods, structures and techniques have not been shown in detail in order not to obscure an understanding of this description.

Given the benefit of the present specification, the skilled person is able to conceive and implement exercise apparatus different to the push down, rowing and fly machines disclosed supra. For example, machines facilitating any of the following types of exercise activity are contemplated: cycling, running, walking, jumping, abdominal crunch, bar throw, bar press, calf press, shoulder shrug, squat, Smith press, elliptical training, gluteus maximus hamstring raise, leg extension, leg press, chest press, bicep curl, lateral raise, shoulder press, triceps extension, pull up, push up, back extension, calf raise, leg curl, stair training, and thigh adductor training are achievable given the guidance provided herein.

As one further example, a stationary bicycle machine may transfer the rotational motion of the crank to a child seat disposed in front of the bicycle such that the child faces the rider. Rotation of the crank may cause rocking of the seat via a rotating plate in a similar manner to that described for preferred embodiment #2 as described herein.

As another example, the pumping action of a stair training machine may be used to alternately inflate and deflate a large air-tight pillow upon which a child reclines. The upward and downward movement of the pillow surface due to inflation and deflation may calm a child while the parent is exercising.

In the following claims, any of the claimed embodiments can be used in any combination.

1. Exercise apparatus configured such that a mechanical motion of a user executing an exercise activity positively affects a child on or about the apparatus.

2. The exercise apparatus according to claim 1 configured to translate a mechanical motion of the user executing an exercise activity on the apparatus into a mechanical motion capable of positively affecting a child on or about the apparatus.

3. The exercise apparatus of claim 1 wherein the positive effect on the child is selected from the group consisting of moving, entertaining, occupying, distracting, stimulating, calming, settling, and educating.

4. The exercise apparatus of claim 1 wherein the mechanical motion capable of affecting a child has the effect of moving a child on or about the apparatus.

5. The exercise apparatus of claim 1 comprising a child physical engagement portion.

6. The exercise apparatus of claim 5 wherein the child physical engagement portion is configured to receive the translated mechanical motion of the user so as to move a child in or about the child physical engagement portion.

7. The exercise apparatus of claim 5 wherein the child physical engagement portion allows a child to be in a seated position.

8. The exercise apparatus of claim 5 wherein the child physical engagement portion comprises a footrest.

9. The exercise apparatus of claim 5 wherein the child physical engagement portion comprises a restraint or a holding portion.

10. (canceled)

11. The exercise apparatus of claim 5 wherein the child physical engagement portion is configured such the child substantially faces the user while the user is executing an exercise activity on the apparatus.

12. The exercise apparatus of claim 5 wherein the child physical engagement portion is configured such that when a child is physically engaged a resistance force is provided against the user executing an exercise activity on the apparatus.

13. The exercise apparatus of claim 1 comprising user movement input portion configured to physically engage with a body part of the user, the movement input portion being moved by the body part of the user executing an exercise activity.

14. The exercise apparatus of claim 13 wherein the user movement input portion is selected from the group consisting of a handle, a bar, a pedal, a chain, a rope, a paddle, a roller, a stirrup, a step, and a tread.

15. The exercise apparatus of claim 13 wherein the user movement input portion is configured to allow the user to move a body part through a range of motion required for an exercise activity.

16. The exercise apparatus of claim 15 wherein the exercise activity is selected from the group consisting of rowing, cycling, running, walking, jumping, abdominal crunch, bar throw, bar press, butterfly, calf press, shoulder shrug, squat, Smith press, elliptical training, gluteus maximus hamstring raise, leg extension, leg press, chest press, bicep curl, lateral raise, shoulder press, triceps extension, pull up, push up, back extension, calf raise, leg curl, stair training, and thigh adductor training.

17. The exercise apparatus of claim 13 comprising child physical engagement portion and a mechanical linkage between the user movement input portion and the child physical engagement portion, the mechanical linkage functioning so as to translate a movement of the user into a movement of the child physical engagement portion.

18. The exercise apparatus of claim 1 comprising a user movement input portion configured to be grasped by at least one hand of the user, the user movement input portion allowing the at least one hand of the user to move bi-directionally substantially vertically when grasping, an elongate member connected to the user movement input portion, child physical engagement portion connected to a region of the member distal to the user movement input portion, wherein upon movement of the movement portion substantially vertically the member translates that movement to the child physical engagement portion such that the child physical engagement portion is concomitantly moved along a vertical plane.

19. The exercise apparatus of claim 1 comprising a user movement input portion configured to be grasped by at least one hand of the user, the user movement input portion allowing the hand(s) of the user to move bi-directionally substantially horizontally when grasping, child physical engagement portion, a mechanical linkage between the user movement input portion and the child physical engagement portion configured such that a substantially horizontal movement of the user movement input portion is translated into motion of the child physical engagement portion.

20. The exercise apparatus of claim 1 comprising a user movement input portion configured to be moved by at least one arms of the user, the user movement input portion allowing the arms(s) of the user to move bi-directionally about at least one shoulder joint, and child physical engagement portion, wherein the child physical engagement portion is attached to the apparatus and configured such that a child disposed in the child physical engagement portion substantially faces the user while the user is executing an exercise activity on the exercise apparatus.

21. (canceled)

22. An exercise facility comprising any two or more different exercise apparatuses, each of the two or more exercise apparatuses according to claim 1.
